(19) United States
(12) Patent Application Publication
(10) Pub. No.: US 2026/0240830 A1
(43) Pub. Date: Aug. 20, 2026

(54) PHOSPHOLIPID COMPLEX CONTAINING ROPIVACAINE OR SALT THEREOF, FORMULATION THEREOF, AND PREPARATION METHOD

(71) Applicant: Beijing Tide Pharmaceutical Co., Ltd., Beijing (CN)

(72) Inventors: Zhihui SU, Beijing (CN); Shaopeng ZHONG, Beijing (CN); Yun WU, Beijing (CN); Boping KANG, Beijing (CN); Qiqi HU, Beijing (CN)

(73) Assignee: Beijing Tide Pharmaceutical Co., Ltd., Beijing (CN)

(21) Appl. No.: 19/385,425

(22) Filed: Nov. 11, 2025

(30) Foreign Application Priority Data
Feb. 14, 2025 (CN) 202510165243.9

(51) Int. Cl. A61K 31/445; A61K 47/10; A61K 47/20; A61K 47/22; A61K 47/24; A61K 47/44

(52) U.S. Cl. CPC A61K 31/445; A61K 47/10; A61K 47/20; A61K 47/22; A61K 47/24; A61K 47/44

(57) ABSTRACT

The present application relates to the field of pharmaceutical formulations, and specifically discloses a phospholipid complex-based ropivacaine sustained-release solution formulation and a preparation method thereof. The formulation has an increased drug loading capacity, good safety, no significant irritation to the administered tissue, and a prolonged sustained-release time.

### CROSS REFERENCE TO RELATED APPLICATION

This application claims the benefit of priority from Chinese Patent Application No. 202510165243.9, filed Feb. 14, 2025, the contents of which are incorporated herein by reference.

### TECHNICAL FIELD

The present application relates to the field of pharmaceutical formulations, and specifically relates to a phospholipid complex-based ropivacaine sustained-release solution formulation and a preparation method thereof.

### BACKGROUND OF THE INVENTION

Postoperative pain refers to pain caused by acute trauma and damage to internal organs and inflammatory stimulation around nerve endings caused by surgical procedures. during the first three days after surgery, patients suffer from the most severe postoperative pain and most likely administrate opioid drugs to control pain. Postoperative pain can have adverse effect on patient's cardiovascular function, respiratory function, neuroendocrine and immune systems, as well as psychological state etc. Currently, postoperative pain is primarily treated with drugs. Clinically used drugs mainly include: opioid analgesics, non-steroidal anti-inflammatory drugs (NSAIDs), and local anesthetics. Among them, local anesthetics exert regional analgesic effects through local nerve blockade, such as bupivacaine and ropivacaine. As an amide-type local anesthetic, ropivacaine is superior to bupivacaine in terms of duration of action, neurotoxicity, and cardiotoxicity, and therefore it is the mainstream local anesthetic choice for anesthesiologists in China.

Ropivacaine injection solution (trade name Naropin) has an onset time of approximately 10 minutes and a short duration of action, lasting only 4-5 hours. However, postoperative pain management usually takes several days, and clinically analgesia is achieved via continuous epidural infusion or intermittent single dose injections. Continuous infusion or long-term indwelling catheters can easily lead to catheter displacement and related complications, and reduces patient compliance. Repeated administration increases fluctuations in blood drug concentration, resulting in systemic adverse effects.

CN104427977B discloses a proliposome, non-aqueous local anesthetic depot formulation, and a preparation method thereof. In the embodiments of the above patent, the highest drug concentration is 4.78% w/w. Using the preparation method disclosed in the patent, the resulting formulation soon precipitates drug crystals when stored at room temperature, and the oil solution turns into a semi-solid gel. That is, according to the embodiments and preparation method disclosed in the patent, a physically stable formulation cannot be obtained. Furthermore, Example 6 of the above patent describes the administration of the depot formulation via subcutaneous injection in a human experimental pain model, and the route of administration is an invasive subcutaneous injection.

CN202410881834.1 discloses a ropivacaine and meloxicam compound phospholipid composition, its preparation method, and use thereof. The phospholipid composition obtained in the invention is lipid vesicles (Example 4, Section 4.4, and FIG. 1). The preparation process for lipid vesicles is complicated and high energy consumption (ultrasonic heating to prepare the solution, sterile filtration, and freeze-drying). The storage stability is poor, requiring storage in lyophilized powder form and reconstitution before use. The medication compliance is poor, as subcutaneous administration is used (in the pharmacodynamic test section of the examples), which is invasive.

Therefore, there is a need to develop a new non-aqueous solution formulation with simple process, low storage requirements, stability at room temperature, non-invasiveness, and topically administration.

### SUMMARY

Based on the above technical background, the present disclosure relates to a phospholipid complex-based ropivacaine sustained-release solution formulation and a preparation method thereof.

After the phospholipid-based sustained-release solution of the present application is administrated to injured tissue, after the solvent diffuses, and the phospholipid encapsulates the drug, forming a solidified precipitate, that provides sustained analgesic effects with the slow release of the drug. Direct topical administration to the injured tissue avoids the use of injection needles, and offers a non-invasive administration method.

The present application discloses a phospholipid complex containing ropivacaine or a salt thereof, wherein, calculated by weight fraction, the ratio range of ropivacaine or the salt thereof to phospholipid is from 1:5 to 1:15, preferably from 1:8 to 1:15, and most preferably from 1:10 to 1:12. As exemplary illustrations, it may include 1:6, 1:7, 1:8, 1:9, 1:10, 1:11, 1:12, 1:13, or 1:14 (calculated as ropivacaine hydrochloride).

In one embodiment of the present application, the phospholipid is selected from natural phospholipids, preferably soybean lecithin, egg yolk lecithin, or a combination thereof.

In one embodiment of the present application, in the phospholipid complex, the content of phosphatidylcholine in the phospholipid is greater than or equal to 94%, preferably from 96.0% to 99.9% (percentage of phospholipid). As exemplary illustrations, it may include 94%, 95%, 96%, 97%, 98%, or 99%.

In one embodiment of the present application, the phospholipid complex further comprises an antioxidant. As one of the embodiments, the antioxidant is selected from cysteine hydrochloride, ascorbic acid, tocopherol, thioglycerol, butylhydroxyanisole, butylated hydroxytoluene, or ascorbyl palmitate, or a combination of two or more thereof, preferably tocopherol.

In one embodiment of the present application, the ropivacaine or the salt thereof is selected from ropivacaine free base, ropivacaine hydrochloride, or ropivacaine mesylate, preferably ropivacaine hydrochloride.

In one embodiment of the present application, the amount of the antioxidant in the phospholipid complex is from 0.02% to 1.8%, preferably from 0.05% to 1.0%, and most preferably 0.05% to 0.2%.

In one embodiment of the present application, the phospholipid complex further comprises an organic solvent, and the organic solvent is selected from anhydrous ethanol, dimethyl sulfoxide, or N-methylpyrrolidone.

In one embodiment of the present application, the amount of the organic solvent in the phospholipid complex is from 5% to 15%, preferably from 7% to 13%, and most preferably from 8% to 10%. As an exemplary illustration, for example, it can be 6%, 7%, 8%, 9%, 10%, 11%, 12%, 13%, 14%, or 15%.

The present application discloses a phospholipid complex-based ropivacaine sustained-release solution formulation, said sustained-release solution formulation, calculated by mass fraction, comprises the following components:

| | |
|---|---|
| Ropivacaine or a salt thereof | 1%~6%; |
| Antioxidant | 0.02%~1.8%; |
| Phospholipid | 30%~72%; |
| Anhydrous ethanol | 5%~15%; and |
| Injection oil | 20% ~50%. |

The ropivacaine sustained-release solution formulation of the present application forms a drug-phospholipid complex, wherein the drug-phospholipid complex is formed by hydrogen bonding between the amide group of ropivacaine and the ester group of phospholipid, thereby increasing the solubility of the drug in the solvent and consequently enhancing the drug concentration in the formulation. The addition of injection oil to the drug-phospholipid complex serves to adjust the solution viscosity and reduce the content of organic solvent in the formulation.

In the present application, as one of the embodiments, the injection oil is selected from soybean oil, castor oil, sesame oil, olive oil, rapeseed oil, sunflower seed oil, corn oil, dipalmitoylphosphatidylglycerol, ethyl oleate, glyceryl dioleate, glyceryl tricaprylate, medium-chain triglycerides, or a combination of two or more thereof. The injection oil may be of natural origin or synthetically produced.

In the present application, the injection oil contains higher aliphatic acids or esters, the higher aliphatic acids or esters include saturated fatty acids or esters and unsaturated fatty acids or esters, with the content of unsaturated fatty acids or esters being higher than that of saturated fatty acids or esters. The content of unsaturated fatty acids or esters is greater than 85%, for example, from 85% to 100%: 86%, 87%, 88%, 89%; or for example, from 90% to 100%: 91%, 92%, 93%, 94%; or for example, from 95% to 100%: 96%, 97%, 98%, 99%.

In the present application, as one of the embodiments, the content of ropivacaine or its salt is from 3% to 5%, and as one of the embodiments, the optimal range is from 3% to 4%.

In the present application, as one of the embodiments, the phospholipid content is from 30% to 39%, for example, 31%, 32%, 33%, 34%, 35%, 36%, 37%, 38%, or 39%.

In the present application, as one of the embodiments, the amount of antioxidant is from 0.05% to 0.2%. As an exemplary illustration may include 0.06%, 0.07%, 0.08%, 0.09%, 0.1%, 0.11%, 0.12%, 0.13%, 0.14%, 0.15%, 0.16%, 0.17%, 0.18%, or 0.19%.

In the present application, as a preferred embodiment, the amount of anhydrous ethanol is from 8% to 10%, and as an exemplary illustration may be 9%.

In the present application, as a preferred embodiment, the amount of injection oil is from 45% to 50%, and as an exemplary illustration may be 46%, 47%, 48%, or 49%.

In the present application, the administration route of the formulation is topical administration. The sustained-release solution formulation can be applied to damaged tissues.

In the present application, as one of the embodiments, the sustained-release solution formulation is selected from any one of the following groups, expressed in mass percentage (w %):

| Component | w % |
|---|---|
| Ropivacaine | 3.0, |
| hydrochloride | |
| Cysteine hydrochloride | 0.1, |
| Anhydrous ethanol | 10.0, |
| Soybean | 38.5, and |
| phosphatidylcholine | |
| Castor oil | 48.4; |
| Ropivacaine | 5.0, |
| hydrochloride | |
| Anhydrous ethanol | 15, |
| Egg yolk | 32.5, and |
| phosphatidylcholine | |
| Castor oil | 47.5; |
| Ropivacaine | 3.2, |
| hydrochloride | |
| Cysteine hydrochloride | 0.1, |
| Anhydrous ethanol | 10.0, |
| Egg yolk | 38.0, and |
| phosphatidylcholine | |
| Castor oil | 48.7; |
| Ropivacaine | 3.2, |
| hydrochloride | |
| Tocopherol | 0.1, |
| Anhydrous ethanol | 10.0, |
| Egg yolk | 38.0, and |
| phosphatidylcholine | |
| Castor oil | 48.7; |

or

| | |
|---|---|
| Ropivacaine | 3.2, |
| hydrochloride | |
| Thioglycerol | 0.1, |
| Anhydrous ethanol | 10.0, |
| Egg yolk | 38.0, and |
| phosphatidylcholine | |
| Castor oil | 48.7. |

The present application further discloses a method for preparing the aforementioned phospholipid complex containing ropivacaine or a salt thereof and formulation thereof. The method comprises the following steps:
(1) mixing ropivacaine or its salt, antioxidant, phospholipid, and anhydrous ethanol, and stirring under heating to dissolve, obtaining a composite solution;
(2) adding injection oil to the composite solution, stirring and mix well, obtaining a clear sustained-release solution;
(3) the sustained-release solution is cooled to room temperature, filled into vials, nitrogen filled, and capped;
(4) sterilizing under high pressure at 0.3-0.4 MPa for 15-20 min, obtaining a sterile formulation.

The present application also discloses another method for preparing the aforementioned formulation, comprising the following steps:
(1) mixing ropivacaine or the salt thereof, the antioxidant, and anhydrous ethanol, and stirring under heating to dissolve, obtaining a clear drug solution;
(2) adding the drug solution from step (1) to phospholipid and injection oil at a temperature ≥60° C., heating at 65° C.-80° C., preferably 70° C.-75° C., and stirring, obtaining a clear sustained-release solution;
(3) the sustained-release solution is cooled to room temperature, and filled into vials, nitrogen filled, and capped;
(4) sterilizing under high pressure at 0.3-0.4 MPa for 15-20 min (such as 18 minutes), obtaining a sterile formulation.

In the aforementioned preparation method of the present application, the heating condition in step (1) is water bath heating at 65° C.-80° C., including water bath heating at 70° C.

The key aspects of the present application are as follows:
(1) Through infrared characterization, it is discovered that the drug and phospholipid are complexed via non-chemical bond, thereby the drug loading capacity is increased up to 6%;
(2) By evaluating both physical and chemical stability of the formulation, antioxidants suitable for this non-polar system are screened;
(3) Direct topical administration to damaged tissues, reduces irritation;
(4) The formulation exhibits sustained-release properties, with a release duration 3.3-6.7 times longer than conventional injections.

The advantages of the present application include:
(1) The drug loading capacity is increased;
(2) Excellent safety profile with no significant irritation to administered tissues;
(3) Prolonged sustained-release duration.

The present application has been validated through animal pharmacokinetic studies and

observations of tissue administration irritation, demonstrating good safety and sustained drug release capability.

In the present application, firstly ropivacaine is combined with phospholipid to prepare a phospholipid complex. Furthermore, by screening antioxidants more suitable for this non-aqueous system, the drug content is increased to 6%. The resulting formulation shows slow impurity growth when it is stored at 60° C. for 30 days, the content of antioxidant is not decreased during the stability test period.

The sustained-release solution obtained in the present application has a viscosity of 600 mPs (25° C.), which facilitates topical administration to damaged tissues, thereby avoiding secondary injury caused by local infiltration injection.

### BRIEF DESCRIPTION OF THE DRAWINGS

FIG. 1: Infrared spectrum of ropivacaine hydrochloride;

FIG. 2: Infrared spectrum of ropivacaine hydrochloride-phospholipid complex.

### DETAILED DESCRIPTION

The following examples are provided to further illustrate the present application but do not limit the effective scope of the invention in any way.

### Example 1: Comparison of Methods for Preparing Ropivacaine Sustained-Release Solution

w %

| | Formu- | Formu- |
|---|---|---|
| Component | lation 1 | lation 2 |
| Ropivacaine | 5.0 | 5.0 |
| hydrochloride | | |
| Anhydrous ethanol | 10.0 | 10.0 |
| Egg yolk | 39.0 | 39.0 |
| phosphatidylcholine | | |
| Castor oil | 46.0 | 46.0 |

### Method for Preparing Formulation 1:

All excipients in the prescribed amounts were added to a vial, stirred in a 75° C. water bath until completely dissolved, followed by adding the prescribed amount of the drug. Heating and stirring were continued until a clear solution was obtained. The solution stands at room temperature, and its appearance was observed.

### Method for Preparing Formulation 2:

The prescribed amounts of ropivacaine hydrochloride, egg yolk phosphatidylcholine, and ethanol were stirred in a 75° C. water bath until they are completely dissolved and clear. The prescribed amount of castor oil was then added, and was stirred until homogeneous. The solution stands at room temperature, and its appearance was observed.

### Results:

Formulation 1 exhibited drug crystal precipitation after one day of storage at room temperature, whereas Formulation 2 remained a clear solution after one month of storage at room temperature. Based on the above results, it is speculated that, during the preparation of Formulation 2, the drug and phospholipid formed a non-chemical bond, thereby improving drug solubility.

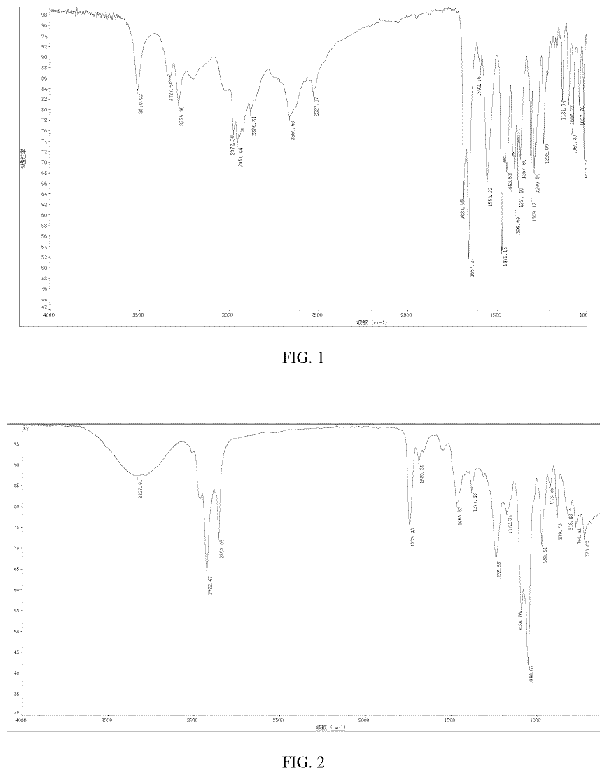

Based on the above results, infrared characterization was performed on the drug and a phospholipid-drug complex prepared at a mass ratio of 1:6.5. The spectra are shown in FIG. 1. The absorption peak of the secondary amide group (1657.37 cm⁻¹) in ropivacaine hydrochloride disappeared or shifted, indicating a non-chemical bond interaction between the amide group of the drug and the phospholipid.

### Example 2: Comparison of Different Phospholipids

Phospholipids included soybean phosphatidylcholine (phosphatidylcholine content>94%), egg yolk phosphatidylcholine (phosphatidylcholine content>96%), and hydrogenated soybean phosphatidylcholine. The formulations were as follows:

w %

| | Formu- | Formu- | Formu- |
|---|---|---|---|
| Component | lation 3 | lation 4 | lation 5 |
| Ropivacaine | 3.0 | 3.0 | 3.0 |
| hydrochloride | | | |
| Cysteine | 0.1 | 0.1 | 0.1 |
| hydrochloride | | | |
| Anhydrous ethanol | 10.0 | 10.0 | 10.0 |
| Soybean | 38.5 | / | / |
| phosphatidylcholine | | | |
| Egg yolk | / | 38.5 | 34.0 |
| phosphatidylcholine | | | |
| Hydrogenated soybean | / | / | 4.5 |
| phosphatidylcholine | | | |
| Castor oil | 48.4 | 48.4 | 48.4 |

### Preparation Method:

The prescribed amounts of ropivacaine hydrochloride, cysteine hydrochloride, phosphatidylcholine, and ethanol were weighed into a vial and heated at 75° C. with stirring until clear. The prescribed amount of castor oil was then added, and was heated and stirred until homogeneous and transparent, the clear solution was obtained and cooled to room temperature, filled into 5 mL vials, nitrogen-filled, and capped.

### Results:

Formulations 3 and 4 yielded clear samples, after Formulation 5 was stirred and mixed well, and then restored to room temperature, material precipitation was observed.

These results demonstrate that natural phospholipids can be used to prepare clear solutions.

### Example 3: Investigation of Drug/Phospholipid Ratio

The solution appearance of different drug/phospholipid ratios (1:6.5-1:12) was investigated. The formulations were as follows:

| | w % | | |
|---|---|---|---|
| | Formu- | Formu- | Formu- |
| Component | lation 6 | lation 7 | lation 8 |
| Ropivacaine | 5.0 | 4.2 | 3.2 |
| hydrochloride | | | |
| Anhydrous ethanol | 15 | 15 | 15 |
| Egg yolk | 32.5 | 38.0 | 38.4 |
| phosphatidylcholine | | | |
| Castor oil | 47.5 | 42.8 | 43.4 |

### Preparation Method:

The prescribed amounts of ropivacaine hydrochloride, egg yolk phosphatidylcholine, and ethanol were weighed into a vial and heated at 70° C. with stirring until a clear solution was obtained. The prescribed amount of castor oil was then added, and the mixture was stirred until homogeneous to obtain a clear and transparent solution. The resulting clear solution was cooled to room temperature, filled into 5 mL vials, nitrogen-filled, capped, and sterilized.

### Results:

All formulations (6-8) yielded clear samples.

### Example 4: Comparison of Different Antioxidants

Since the present product contains oxidation-prone oily excipients and involves a heating process, the types of antioxidants were screened. Cysteine hydrochloride, tocopherol, and thioglycerol were selected to prepare solutions and investigate product stability. The formulations were as follows:

| | w % | | | | |
|---|---|---|---|---|---|
| | Formu- | Formu- | Formu- | Formu- | Formu- |
| Component | lation 9 | lation 10 | lation 11 | lation 12 | lation 13 |
| Ropivacaine | 3.2 | 3.2 | 3.2 | 3.2 | 3.2 |
| hydrochloride | | | | | |
| Cysteine | 0.1 | / | / | / | / |
| hydrochloride | | | | | |
| Tocopherol | / | 0.1 | / | / | / |
| Thioglycerol | / | / | 0.1 | 0.3 | 0.5 |
| Anhydrous | 10.0 | 10.0 | 10.0 | 10.0 | 10.0 |
| ethanol | | | | | |
| Egg yolk | 38.0 | 38.0 | 38.0 | 38.0 | 38.0 |
| phosphati- | | | | | |
| dylcholine | | | | | |
| Castor oil | 48.7 | 48.7 | 48.7 | 48.5 | 48.3 |

### Preparation Method:

The prescribed amounts of ropivacaine hydrochloride, antioxidant, and ethanol were weighed into a vial. The mixture was heated and stirred at 70° C. until clear. The prescribed amounts of egg yolk phosphatidylcholine and castor oil were added, and heated and stirred well until a clear and transparent solution was obtained. The solution was cooled to room temperature, filled into 5 mL vials, nitrogen filed, capped, and sterilized. The above samples stand at 60° C., and sampling was performed at 10 days and 30 days to determine drug content, related substances, carbonyl value, and acid value.

### Stability Results are as Follow:

| | Formulation 9 | | Formulation 10 | | Formulation 11 | | Formulation 12 | | Formulation 13 | |
|---|---|---|---|---|---|---|---|---|---|---|
| | 60° C.- | 60° C.- | 60° C.- | 60° C.- | 60° C.- | 60° C.- | 60° C.- | 60° C.- | 60° C.- | 60° C.- |
| | 10 d | 10 d | 10 d | 10 d | 10 d | 10 d | 10 d | 10 d | 10 d | 10 d |
| Content | 98.2% | 99.2% | 99.0% | 98.8% | 100.4% | 100.5% | 100.2% | 100.8% | 100.7% | 100.7% |
| Total content | 0.13% | 0.24% | 0.17% | 0.16% | 0.12% | 0.15% | 0.08 | 0.20% | 0.07 | 0.16% |
| of Related | | | | | | | | | | |
| substances | | | | | | | | | | |
| Carbonyl Value | 5.39 | 6.86 | 6.29 | 7.34 | 7.54 | 7.97 | 7.08 | 7.06 | 7.09 | 7.10 |
| Acid Value | / | 12.5 | / | 9.0 | / | 12.3 | / | 9.3 | / | 10.1 |

Based on the results, it was observed that Formulations 9 to 13, when stored at 60° C. for 30 days, showed no reduction of drug content and low total impurities. Compared to other formulations, Formulation 11 had a higher carbonyl value, and a high carbonyl value typically indicated a higher degree of lipid oxidation, which suggests that harmful substances such as aldehydes and ketones have been produced in oils and fats. After 30 days at 60° C., Formulations 9, 11, and 13 showed higher acid values than Formulations 10 and 12, and acid value reflects the degree of lipid rancidity. Based on the comprehensive evaluation of related substances, carbonyl value, and acid value, Formulation 10 was the optimal formulation.

### Example 5: Preliminary Pharmacokinetic Study in Beagle Dogs

### Materials and Methods:

A total of 24 healthy Beagle dogs (half male and half female) were used in this study. The animals were randomly divided into 3 groups, with 8 dogs in each group: a positive control group, Formulation Group 1 (Formulation 7), and Formulation Group 2 (Formulation 9). On the day of surgery, the animals were anesthetized, and a longitudinal incision of approximately 5-6 cm was made on the inner thigh of one side. After cutting through the skin and subcutaneous fascia, a longitudinal incision of about 2 cm depth was made in the inner thigh muscle group at the incision. After the incision is completed, each animal in Formulation Group 1 and Formulation Group 2 were topically administered the corresponding drug solutions into the wound. The positive control group received no treatment. Subsequently, the muscle and skin of all animals were sutured, and the wound was disinfected with iodine. The positive control group was administered the corresponding ropivacaine hydrochloride injection subcutaneously at multiple sites after suturing. The total drug dose was consistent between the formulation groups and the positive control group.

Blood samples from all animals in the positive control group were collected before administration and at 15 min, 30 min, 1 h, 2 h, 4 h, 6 h, 8 h, and 24 h after administration. Blood samples from all animals in Formulation Group 1 and Formulation Group 2 were collected before administration and at 30 min, 1 h, 2 h, 4 h, 6 h, 8 h, 24 h, 32 h, 48 h, 72 h, and 96 h after administration. Subsequently, the drug concentration (measuring ropivacaine), elimination half-life (t½), area under the blood concentration-time curve (AUC0-∞), peak concentration (Cmax), and time to peak concentration (Tmax) were measured at each time point. The mean residence time (MRT0-∞) was calculated.

### Results are Shown as Follow:

| | | T_{1/2} | Tₘₐₓ | Cₘₐₓ | AUC_{INF} | MRTₗₐₛₜ |
|---|---|---|---|---|---|---|
| | | (h) | (h) | (ng/mL) | (h*ng/mL) | (h) |
| Gender | Group | Mean | Mean | Mean | Mean | Mean |
| Female | Positive | 2.81 | 0.33 | 4015 | 11303 | 3.76 |
| | Control | | | | | |
| | Formulation | 30.5 | 4.05 | 412 | 5784 | 12.5 |
| | Group 1 | | | | | |
| | Formulation | 30.8 | 4.12 | 402 | 5776 | 12.8 |
| | Group 2 | | | | | |
| Male | Positive | 2.47 | 0.38 | 2610 | 5144 | 1.75 |
| | Control | | | | | |
| | Formulation | 33.7 | 4.00 | 498 | 5645 | 11.2 |
| | Group 1 | | | | | |
| | Formulation | 34.2 | 4.10 | 512 | 5573 | 11.7 |
| | Group 2 | | | | | |

Compared with the positive control group (subcutaneous multi-point injection of ropivacaine hydrochloride injection, Naropin, manufactured by AstraZeneca AB), the T½, Tmax, and MRTlast of ropivacaine in vivo were prolonged after topical administration of the same dose of formulation solutions into the wound, indicating a sustained-release characteristic of the formulation groups at the local administration site. The sustained-release duration of the formulation groups was 3.3-6.7 times that of the positive control group.

### Example 6 Observation of Local Irritation at Animal Administration Sites

During the pharmacokinetic study in Example 4, the surgical wound sites of animals in each group were observed to evaluate local irritation of different administration groups. The results are shown in the table below:

### Observation of Administration Sites of Dogs

| | Animals | Symptoms/ | Observation |
|---|---|---|---|
| Group | No. | Score | Time |
| Model group | 1M001 | Visible edema/ | D1~D4 |
| | | 2 scores | |
| | 1M002 | Barely visible | D3 |
| | | edema/1 score | |
| Formulation | 2F004 | Barely visible | D3 |
| Group 2 | | edema/1 score | |

Note:
Edema score 1 = barely visible edema; Edema score 2 = visible edema.

The visual observation of skin irritation reaction at the administration sites revealed that only animal 1M001 exhibited visible edema on the fourth day post-surgery; 1M002 showed barely visible edema on the third day post-surgery; and 2F004 displayed barely visible edema on the third day post-surgery. No abnormal irritant reactions were observed at the administration sites in any other animals. Only a few animals exhibited mild skin irritation, which was considered unrelated to the test substance.

These above observations indicate that topical administration causes less irritation compared to subcutaneous multi-point injections.

What is claimed is:

1. A phospholipid complex containing ropivacaine or a salt thereof, calculated by mass fraction, wherein the weight ratio of the ropivacaine or the salt thereof to the phospholipid is 1:5 to 1:15, preferably 1:8 to 1:15; and most preferably 1:10 to 1:12.

2. The phospholipid complex according to claim 1, wherein the phospholipid is selected from natural phospholipids; preferably selected from soybean lecithin, egg yolk lecithin, or a combination thereof.

3. The phospholipid complex according to claim 1, wherein the weight percentage of phosphatidylcholine in the phospholipid is greater than or equal to 94%; preferably 96.0% to 99.9%.

4. The phospholipid complex according to claim 1, wherein the phospholipid complex further comprises an antioxidant, and the antioxidant is selected from cysteine hydrochloride, ascorbic acid, tocopherol, thioglycerol, butylhydroxyanisole, butylated hydroxytoluene, or ascorbyl palmitate, or a combination of two or more thereof; preferably tocopherol.

5. The phospholipid complex according to claim 1, wherein the ropivacaine or the salt thereof is selected from ropivacaine free base, ropivacaine hydrochloride, or ropivacaine mesylate, preferably ropivacaine hydrochloride.

6. The phospholipid complex according to claim 4, wherein the weight percentage of the antioxidant is 0.02% to 1.8%, preferably 0.05% to 1.0%, and most preferably 0.05% to 0.2%.

7. The phospholipid complex according to claim 1, wherein the phospholipid complex comprises an organic solvent, and the organic solvent is selected from anhydrous ethanol, dimethyl sulfoxide, or N-methylpyrrolidone.

8. The phospholipid complex according to claim 7, wherein the amount of the organic solvent is 5% to 15%, preferably 7% to 13%, and most preferably 8% to 10%.

9. The phospholipid complex according to claim 1, wherein the ropivacaine or the salt thereof is selected from ropivacaine free base, ropivacaine hydrochloride, or ropivacaine mesylate, preferably ropivacaine hydrochloride.

10. A ropivacaine sustained-release solution formulation containing the phospholipid complex according to claim 1, wherein the sustained-release solution formulation comprises, calculated by mass fraction:

| Component | w % |
|---|---|
| Ropivacaine or a salt thereof | 1%~6%; |
| Antioxidant | 0.02%~1.8%; |
| Phospholipid | 30%~72%; |
| Anhydrous ethanol | 5%~15%; and |
| Injection oil | 20%~50%. |

11. The formulation according to claim 10, characterized in that, the injection oil is selected from soybean oil, castor oil, sesame oil, olive oil, rapeseed oil, sunflower seed oil, corn oil, dipalmitoylphosphatidylglycerol, ethyl oleate, diolein, tricaprylin, or medium-chain triglycerides, or a combination of two or more thereof, and the injection oil is of natural origin or synthetically produced.

12. The formulation according to claim 11, characterized in that, the injection oil contains higher aliphatic acids or esters, the higher aliphatic acids or esters include saturated fatty acids or esters and unsaturated fatty acids or esters, and the content of unsaturated fatty acid esters is higher than that of saturated fatty acid esters, with the content of unsaturated fatty acids or esters being from 85% to 100%.

13. The formulation according to claim 10, wherein the weight percentage of the ropivacaine or the salt thereof is from 3% to 5%, most preferably from 3% to 4%.

14. The formulation according to claim 10, wherein the weight percentage of the phospholipid is 30% to 39%.

15. The formulation according to claim 10, wherein the weight percentage of the antioxidant is 0.05% to 0.2%.

16. The formulation according to claim 10, wherein the weight percentage of the anhydrous ethanol is 8% to 10%.

17. The formulation according to claim 10, wherein the weight percentage of the injection oil is 45% to 50%.

18. The formulation according to claim 10, characterized in that, an administration route of the formulation is topical administration.

19. The formulation according to claim 10, wherein the sustained-release solution formulation is selected from any one of the following groups, calculated by mass fraction:

| Component | w % |
|---|---|
| Ropivacaine hydrochloride | 3.0, |
| Cysteine hydrochloride | 0.1, |
| Anhydrous ethanol | 10.0, |
| Soybean phosphatidylcholine | 38.5, and |
| Castor oil | 48.4; |
| Ropivacaine hydrochloride | 5.0, |
| Anhydrous ethanol | 15, |
| Egg yolk phosphatidylcholine | 32.5, and |
| Castor oil | 47.5; |
| Ropivacaine hydrochloride | 3.2, |
| Cysteine hydrochloride | 0.1, |
| Anhydrous ethanol | 10.0, |
| Egg yolk phosphatidylcholine | 38.0, and |
| Castor oil | 48.7; |
| Ropivacaine hydrochloride | 3.2, |
| Tocopherol | 0.1, |
| Anhydrous ethanol | 10.0, |
| Egg yolk phosphatidylcholine | 38.0, and |
| Castor oil | 48.7; |

or

| | |
|---|---|
| Ropivacaine hydrochloride | 3.2, |
| Thioglycerol | 0.1, |
| Anhydrous ethanol | 10.0, |
| Egg yolk phosphatidylcholine | 38.0, and |
| Castor oil | 48.7. |

20. A method for preparing the formulation according to claim 10, comprising the following steps:
(1) mixing ropivacaine or its salt, antioxidant, phospholipid, and anhydrous ethanol, and stirring under heating to dissolve, obtaining a composite solution;
(2) adding injection oil to the composite solution, stirring to mix well, obtaining a clear sustained-release solution;
(3) the sustained-release solution is cooled to room temperature, filled into vials, nitrogen filed, and capped;
(4) sterilizing under high pressure at 0.3-0.4 MPa for 15-20 min, obtaining a sterile formulation;
preferably, wherein the method comprises the following steps:
(1) mixing ropivacaine or the salt thereof, the antioxidant, and anhydrous ethanol, and stirring under heating to dissolve, obtaining a clear drug solution;
(2) adding the drug solution from step (1) to phospholipid and injection oil at a temperature ≥60° C., heating at a temperature of 65° C.-80° C., preferably 70° C.-75° C., and stirring, obtaining a clear sustained-release solution;
(3) the sustained-release solution is cooled to room temperature, filled into vials, nitrogen filled, and capped;
(4) sterilizing under high pressure at 0.3-0.4 MPa for 15-20 min, obtaining a sterile formulation.

21. The method according to claim 20, wherein the heating conditions in step (1) is 65° C.-80° C. water bath heating.
